(19) United States
(12) Patent Application Publication
(10) Pub. No.: US 2025/0251330 A1
(43) Pub. Date: Aug. 7, 2025

(54) ANALYSIS DEVICE AND METHOD FOR ANALYZING A VISCOSITY OF A FLUID

(71) Applicant: KARDION GMBH, Stuttgart (DE)

(72) Inventors: Ingo Stotz, Ditzingen (DE); Thomas Alexander Schlebusch, Renningen (DE); Tobias Schmid, Stuttgart (DE)

(21) Appl. No.: 19/049,690

(22) Filed: Feb. 10, 2025

Related U.S. Application Data
(63) Continuation of application No. 15/734,489, filed on Jul. 20, 2021, now Pat. No. 12,222,267.

(30) Foreign Application Priority Data
Jun. 6, 2018 (DE) 102018208945.0

(51) Int. Cl. G01N 11/04; A61M 60/174; A61M 60/216; A61M 60/422; G01N 11/00; G01N 33/49

(52) U.S. Cl. CPC G01N 11/04; A61M 60/174; A61M 60/216; A61M 60/422; G01N 33/49; G01N 2011/0073

(57) ABSTRACT

The approach presented here relates to an analysis device for analyzing a viscosity of a fluid. The analysis device comprises a detection device and a provisioning device. The detection device is formed to determine the viscosity of the fluid using at least one Doppler parameter of a Doppler spectrum of the fluid. The provisioning device is formed to provide or transmit a viscosity signal that represents the viscosity determined by the detection device.

### INCORPORATION BY REFERENCE TO ANY PRIORITY APPLICATIONS

The present application is a continuation of U.S. application Ser. No. 15/734,489 filed on Jul. 20, 2021, which is a National State entry of PCT Patent Application No. PCT/EP2019/064810, filed on Jun. 6, 2019, titled ANALYSIS APPARATUS AND METHOD FOR ANALYZING A VISCOSITY OF A FLUID, which is an International Application of and claims the benefit of priority to German Patent Application No. 102018208945.0, filed on Jun. 6, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND

### Field

The invention relates to an analysis device and a method for analyzing a viscosity of a fluid. The invention also relates to a computer program and a machine-readable storage medium on which the computer program is stored.

### Description of the Related Art

PT (prothrombin time) and INR (International Normalized Ratio) are the standard measure for blood coagulation. Usually, the INR in blood samples is determined by adding thromboplastin and then measuring the time to coagulation. The determination can be carried out in the laboratory; meanwhile, test strip devices are now also available for self-measurement by the patient, comparable to the procedure of a blood sugar measurement. Coagulation management is essential for patients with cardiac assist systems to minimize pump thrombosis. Monitoring of blood viscosity as an INR substitute parameter may be sufficient for coagulation management.

EP 2,175,770 B1 describes an explicit blood viscosity sensor based on surface waves, abbreviated as SAW, for determining viscosity.

U.S. Pat. No. 7,591,777 B2 describes a viscosity determination in cardiac assist systems by the mechanical effect of the blood viscosity on the drive of the cardiac assist system.

### SUMMARY

The task of the invention is to provide an improved method for analyzing a viscosity of a fluid and an improved analysis device for this purpose. In particular, it is a task of the invention to specify a method and a device that allows the viscosity of a fluid to be analyzed continuously and on a short time scale.

This task is achieved by the determination devices, systems, and methods disclosed herein. Advantageous embodiments of the invention are disclosed herein.

An analysis device for analyzing a viscosity of a fluid and a method according to the invention for analyzing a viscosity of a fluid and finally a corresponding computer program are presented below. Advantageous further embodiments and improvements of the subject matter specified herein are possible using the measures specified herein.

In light of this background, the approach presented here presents an analysis device for analyzing a viscosity of a fluid and a method for analyzing a viscosity of a fluid and finally a corresponding computer program according to the main claims. Advantageous further developments and improvements of the device specified herein are possible using the measures listed herein.

The advantages achievable with the presented approach are that an analysis device presented here is designed to determine and provide or transmit the viscosity of a fluid quickly and easily using a real-time Doppler parameter of the fluid. A Doppler parameter can in this case be understood to mean a parameter that represents information about a change in a frequency of a signal emitted into the fluid to a frequency of a signal received from the fluid. For example, the Doppler parameter corresponds to a Doppler shift. In the present case, a Doppler spectrum can be understood to mean a spectrum that contains frequencies that result from a signal emitted into the fluid, as well as frequencies that result from a signal received from the fluid. This approach then can for example permit an analysis of the Doppler shift of different frequency components of signals emitted into the fluid with respect to the frequency components resulting from signals received from the fluid.

An analysis device for analyzing a viscosity of a fluid is presented. The analysis device comprises a detection device and a provisioning device. The detection device is formed to determine the viscosity of the fluid using at least one Doppler parameter of a Doppler spectrum of the fluid. The provisioning device is formed to provide or emit a viscosity signal that represents the viscosity determined by the detection device. The Doppler spectrum is to be understood as a product from a flow profile of the fluid and a directional characteristic of an ultrasonic element, which generates or can generate a sound wave in the fluid. The flow profile can be dependent on a flow velocity of the fluid and additionally or alternatively on a shaping of an intake device through which the fluid flows.

The detection device can be designed to read the Doppler parameter from such an ultrasonic element, which can be an ultrasonic transducer. The ultrasonic element can be formed to generate the sound wave in the fluid and to sense the Doppler parameter of a returning reflected sound wave in the fluid. The generated sound wave can have a defined or fixed directional characteristic. The detection device and/or the provisioning device can be part of, or be formed to be coupled to, the ultrasonic element. For example, the detection device can be formed to read the Doppler parameter sensed by the ultrasonic element from the ultrasonic element.

The detection device can be formed to determine the viscosity using a functional relationship between the Doppler parameter to the viscosity and/or using a lookup table, in particular wherein a relationship between the Doppler parameter to the viscosity can be stored in the lookup table. The look-up table can be a calibration table that can store measurement data for all relevant viscosities of the fluid for all relevant Doppler parameters and additionally or alternatively other relevant parameters such as flow velocities of the fluid. Using the real-time Doppler parameter, a viscosity mapped thereto can then be read quickly and easily from the look-up table. Or the viscosity can be quickly and easily determined by solving the functional relationship using the real-time Doppler parameter. The lookup table and/or the functional relationship can be stored in the detection device or can be read for use by the detection device.

It is also advantageous if the detection device is formed according to an embodiment to determine the viscosity using an interpolation of a first viscosity stored in the lookup table and a second (adjacent) viscosity stored in the lookup table. This allows calculation accuracy to be increased.

The analysis device can also comprise a cannula having an intake interface for receiving the fluid and an outlet interface opposite the intake interface for discharging the fluid, in particular wherein the Doppler parameter can represent a Doppler parameter in the cannula. Such a cannula can be formed for use on or in a cardiac assist system. For example, the cannula can be shaped or formed to receive blood as the fluid. The real-time viscosity of the blood in the cannula can then be advantageously determined using the analysis device. The detection device can also be formed to determine the viscosity using at least one cannula parameter of the cannula. The cannula parameter can be a cannula width or a cannula radius.

According to a further advantageous embodiment, the analysis device comprises a flow device for conveying the fluid from the intake interface to the outlet interface of the cannula, in particular wherein the flow device can be arranged or arrangeable on or in the area of the outlet interface. The flow device can comprise a drive device in the form of an electric motor and a coupled impeller. When the flow device is in operation, a volume flow of the fluid can thus be caused through the cannula, wherein the volume flow renders the flow profile measurable as a function of the viscosity of the fluid, a flow velocity of the fluid, and a shaping of the cannula, for example the cannula width or the cannula radius. Such an analysis device with a flow device can be formed or usable as a cardiac assist system. This cardiac assist system can advantageously determine a real-time blood viscosity and provide or transmit it for example for a diagnostic method.

The detection device can also be formed to determine the viscosity using at least one flow parameter of the flow profile, in particular a flow velocity, of the fluid through the cannula. The flow velocity can be measurable using an ultrasonic element formed to sense the Doppler shift of the ultrasonic signal reflected on particles of the fluid.

It is further advantageous if the analysis device according to an exemplary embodiment comprises an ultrasonic element, which is formed to generate a sound wave in the fluid in order to detect the Doppler parameter, in particular wherein the ultrasonic element can be arranged in the region of the intake interface of the cannula. The ultrasonic element can be formed to generate the sound wave with a defined or fixed directional characteristic. In this case, the directional characteristic can be aligned in the direction of the expected fluid flow of the fluid through the cannula.

The detection device can be formed to determine the viscosity using the Doppler parameter, which represents a Doppler frequency and/or a width of the Doppler spectrum.

A method for analyzing a viscosity of a fluid is also presented. The method comprises a detection step and a provisioning step. The detection step involves determining the viscosity of the fluid using at least one Doppler parameter of a Doppler spectrum of the fluid. The provisioning step involves providing or transmitting a viscosity signal, which represents the viscosity determined during the detection step.

This method can be performed using the analysis device presented above. The method can be implemented in software or hardware, for example, or in a mixed form of software and hardware, for example in a control device.

A computer program product or computer program having program code which can be stored on a machine-readable carrier or storage medium such as a semiconductor memory, a hard drive memory, or optical memory and is used to carry out, implement, and/or control the steps of the method according to one of the embodiments described above is also advantageous, in particular if the program product or program is executed on a computer or a device.

### BRIEF DESCRIPTION OF THE DRAWINGS

Design examples of the approach presented here are shown in the drawings and explained in more detail in the following description. The drawings show in:

FIG. 1 is a schematic illustration of an analysis device for analyzing a viscosity of a fluid according to an exemplary embodiment;

FIG. 2 is a schematic cross-sectional side view illustration of an analysis device according to an exemplary embodiment;

FIG. 3 is a schematic illustration of a cardiac assist system with an analysis device according to an exemplary embodiment;

FIG. 4 is a schematic illustration of a flow profile of a fluid according to an exemplary embodiment;

FIG. 5 is a schematic illustration of a Doppler spectrum;

FIG. 6 is a schematic illustration of a Doppler spectrum;

FIG. 7 is a schematic illustration of a Doppler spectrum according to an exemplary embodiment; and

FIG. 8 is a flow diagram of a method for analyzing a viscosity of a fluid according to an exemplary embodiment.

### DETAILED DESCRIPTION

The following description of favorable exemplary embodiments of the present invention uses the same or similar reference symbols shown in the various figures for elements that act in similar ways, wherein a repeated description of these elements is omitted.

If a design example includes an “and/or” conjunction between a first feature and a second feature, this should be read to mean that the design example according to one embodiment comprises both the first feature and the second feature and, according to another embodiment, comprises either only the first feature or only the second feature.

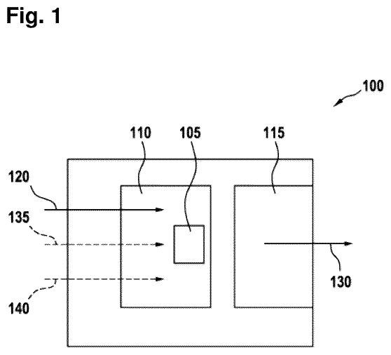

FIG. 1 shows a schematic illustration of an analysis device 100 for analyzing a viscosity 105 of a fluid according to an exemplary embodiment.

The analysis device 100 comprises a detection device 110 and a provisioning device 115. The detection device 110 is formed to detect the viscosity 105 of the fluid using at least one Doppler parameter 120 of a Doppler spectrum of the fluid. The provisioning device 115 is formed to provide or transmit a viscosity signal 130 representing the viscosity 105 determined by the detection device 110.

According to this exemplary embodiment, the detection device 110 is designed to determine the viscosity 105 using a flow parameter 135 of the fluid through a cannula, in which the fluid is accommodated, and/or to determine a cannula parameter 140 of the cannula. According to this exemplary embodiment, the detection device 110 is formed to read the Doppler parameter 120 and/or the flow parameter 135 and/or the cannula parameter 140 in the form of a sensor signal each.

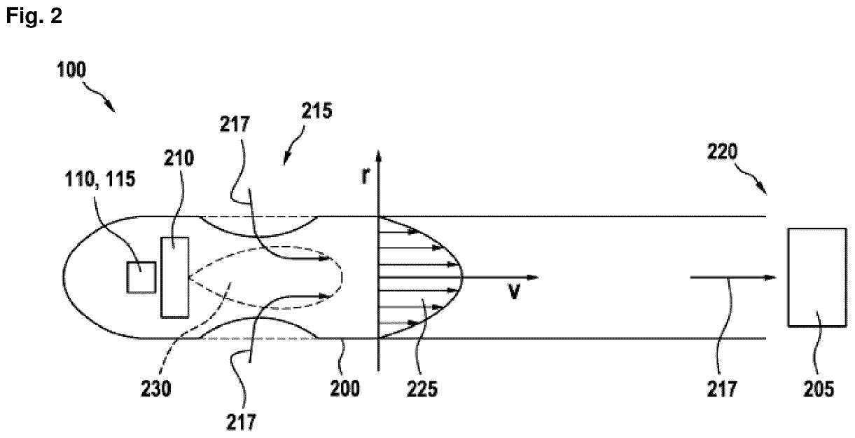

FIG. 2 shows a schematic cross-sectional side view of an analysis device 100 according to an exemplary embodiment. This can be the analysis device 100 described in FIG. 1, with the difference being that the analysis device 100 according to this exemplary embodiment additionally comprises a cannula 200, a flow device 205 and an ultrasonic element 210. Alternatively or additionally, the analysis device 100 can for example be formed as two components so that the cannula 200, the flow device 205 and the ultrasonic element 210 can be operated spatially separated from the detection device 110 and the provision device 115 using a cable.

The cannula 200 has an intake interface 215 formed to receive the fluid 217 and an outlet interface 220 formed to discharge the fluid 217 opposite the intake interface 215. According to this exemplary embodiment, the Doppler parameter represents a Doppler parameter in the cannula 200.

The flow device 205 is formed to convey the fluid 217 from the intake interface 215 to the outlet interface 220 of the cannula 200. For this purpose, the flow device 205 according to this exemplary embodiment is arranged or can be arranged on or in the area of the outlet interface 220. According to this exemplary embodiment, the flow device 205 comprises a drive device in the form of an electric motor and/or a coupled impeller, which is accommodated in the cannula 200.

According to this exemplary embodiment, the detection device 110 is formed to determine the viscosity using the flow parameter, which represents a flow velocity v of a flow profile 225 of the fluid through the cannula 200. According to this exemplary embodiment, the detection device 110 is also formed to determine the viscosity using the cannula parameter of the cannula 200, which represents a cannula width r of the cannula 200.

The ultrasonic element 210 is formed to generate a sound wave in the fluid 217 in order to determine the Doppler parameter in the reflection of the sound waves on particles in the fluid.

According to this exemplary embodiment, the ultrasonic element 210 is arranged in the region of the intake interface 215 of the cannula 200. A directional characteristic 230 of the ultrasonic element 210 is also shown, wherein the directional characteristic 230 is fixed and/or defined according to this exemplary embodiment.

According to this exemplary embodiment, the detection device 110 is formed to determine the viscosity using the Doppler parameter, which represents a Doppler frequency and/or a width of the Doppler spectrum. According to this exemplary embodiment, the detection device 110 is formed to determine the viscosity using a functional relationship between the Doppler parameter to the viscosity and/or using a lookup table, wherein a relationship between the Doppler parameter and the viscosity is stored in the lookup table. According to this exemplary embodiment, the detection device 110 is also formed to determine the viscosity by using an interpolation of a first viscosity stored in the lookup table and an adjacent second viscosity stored in the lookup table.

The following again describes details of the analysis device 100 in more detail and in other words:

According to this exemplary embodiment, the analysis device 100 presented here can be used as a cardiac assist system. For patients with a cardiac assist system, also called VAD patients, where VAD stands for “Ventricular Assist Device”, coagulation management is essential to minimize pump thrombosis. For this purpose, patients are for example treated with drugs for inhibiting plasma blood coagulation, and the INR is thus for example adjusted in the range 2 to 2.5.

The flow profile 225 and thus the viscosity of the blood can be determined by analyzing the Doppler spectrum with the ultrasonic element 210 integrated according to this exemplary embodiment in a tip of the cannula 200 of a VAD system, which can also be referred to as an inlet cannula.

In accordance with this exemplary embodiment, the blood viscosity is determined by the detection device 110 while the analysis device 100 is in operation, either continuously or at fixed time intervals in accordance with an alternative exemplary embodiment. The provisioning device 115 is formed to provide a physician and/or patient with the determined viscosity as a parameter for therapy management. For this purpose, the viscosity signal is formed to display the viscosity on a display and/or to transmit it to a web service by wireless transmission.

Advantageously, in the analysis device 100 presented here, only a simple so-called “single element” ultrasonic transducer is sufficient as an ultrasonic element 210, which is formed according to this exemplary embodiment as a circular disk. Such an ultrasonic element 210 is possible due to the special spatial positioning of the ultrasonic element 210 shown here in the direction of the expected flow of the fluid 217. The ultrasonic element 210 is formed according to an exemplary embodiment for quantifying the flow velocity v of the fluid 217.

The ultrasonic element 210 integrated in the tip of the intake cannula measures the Doppler spectrum of the flow in the cannula 200, for example with the so-called “pulsed-wave Doppler” method; this method is also called a “pulsed Doppler”.

In other words, FIG. 2 shows an exemplary embodiment of a VAD intake cannula with ultrasonic element 210 in the form of an ultrasonic transducer. FIG. 2 shows an intake region, the directional characteristic 230 of the ultrasonic transducer, and the adjusting flow profile 225 in the intake cannula.

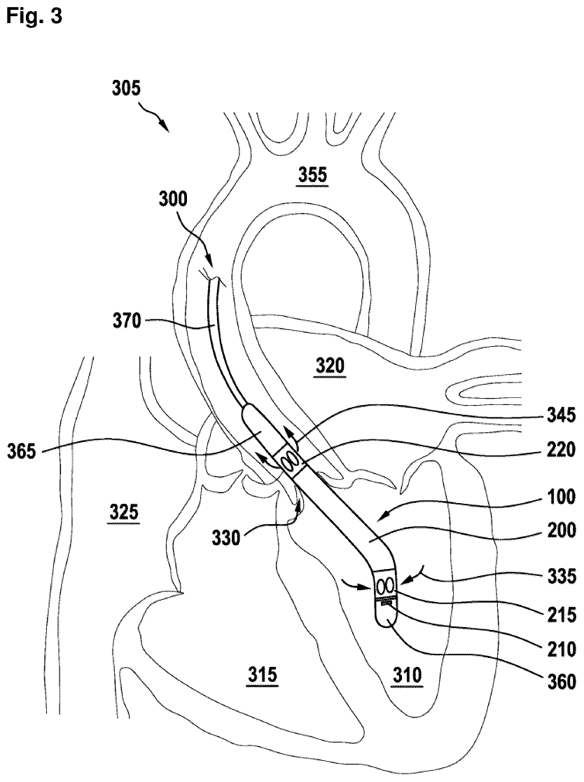

FIG. 3 shows a schematic illustration of a cardiac assist system 300 with an analysis device 100 according to an exemplary embodiment. This can be the analysis device 100 described with reference to FIG. 2.

The cardiac assist system 300 shown here as an example can also be referred to as a cardiac assist system. FIG. 3 also shows a heart 305 with left ventricle 310 and right ventricle 315 as well as left atrium 320 and right atrium 325. The cardiac assist system 300 is located in the center of the aortic valves 330, so that a blood stream 335 is suctioned through the intake interface 215 in the form of intake openings in the region of the left ventricle 310, and is discharged into the aorta 355 in the region downstream of the heart valves 345 through the outlet interface 220 in the form of outlet openings.

According to this exemplary embodiment, the assist system also comprises a distal tip 360 with sensors; according to an exemplary embodiment, the sensors comprise at least one pressure and/or at least one temperature sensor, as well as the ultrasonic element 210, which radiates into the cannula 200 along the axis of the support system through an intake region of the intake interface 215. The cannula 200 directs the blood to the flow machine with impeller, which is located in the area of the outlet interface 220. This is followed by an electric motor 365 and a connection cable 370.

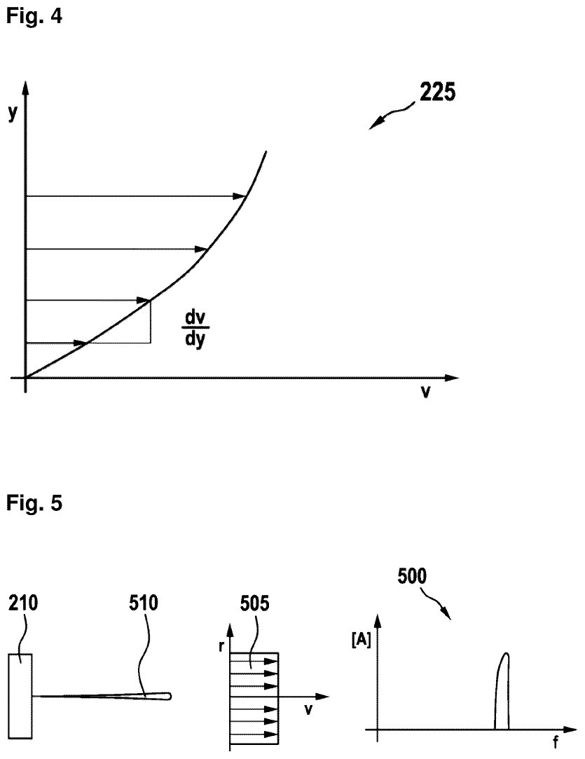

FIG. 4 shows a schematic illustration of a flow profile 225 of a fluid according to an exemplary embodiment. This can be the flow profile 225 described in FIG. 2, which can be determined by one of the analysis devices described in one of the preceding figures. An exemplary flow profile 400 is shown in a tube, wherein v denotes a velocity of the fluid and y a radial distance from a tube inner wall of the tube. The velocity gradient [symbol] v/ [symbol] y, and thus the velocity profile, is viscosity-dependent. In other words, the velocity profile in a cannula of a cardiac assist system according to Navier-Stokes is dependent on the viscosity.

FIG. 5 shows a schematic illustration of a Doppler spectrum 500. The Doppler spectrum 500 is the product of a flow profile 505 of a fluid and a directional characteristic 510 of an ultrasonic element 210. FIGS. 5 to 7 compare different flow profiles and directional characteristics as well as respectively resulting Doppler spectra, wherein FIG. 7 shows a real Doppler spectrum in the manner effected and/or discernable using the analysis devices presented in any of FIGS. 1 to 3.

FIG. 5 shows a Doppler spectrum 500 for an ideally focusing ultrasonic element 210, which causes an ideal directional characteristic 510, and a parallel flow, which results in the parallel flow profile 505.

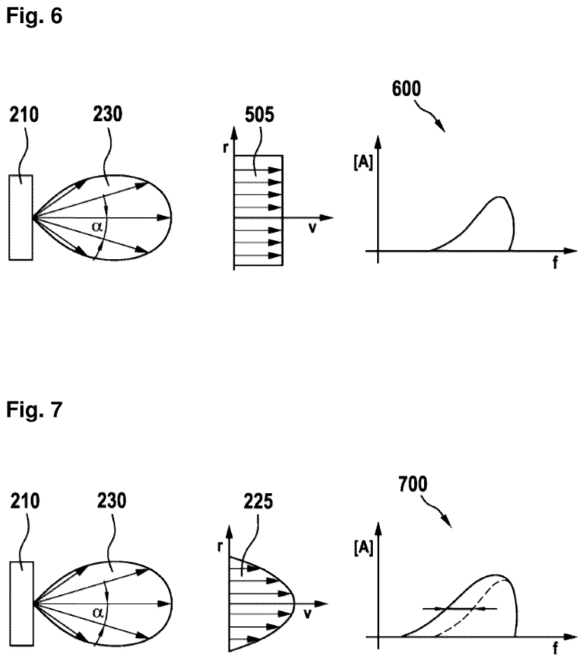

FIG. 6 shows a schematic illustration of a Doppler spectrum 600. The figure shows a resulting Doppler spectrum 600 for a real focusing ultrasonic element 210, which causes the directional characteristic 230 described for use with the analytical device described in any of FIGS. 1 to 3, and the parallel flow profile 505 described in FIG. 5. Compared to the Doppler spectrum shown in FIG. 5, the Doppler spectrum 600 resulting in FIG. 6 is widened.

FIG. 7 shows a schematic illustration of a Doppler spectrum 700 according to an exemplary embodiment. This can be the Doppler spectrum 700, as is caused and/or discernible in the cannula using the analysis devices shown in any of FIGS. 1 to 3.

The figure shows a resulting Doppler spectrum 700 of the fluid for the real focusing ultrasonic element 210, which has a real directional characteristic 230, and a real flow profile 225 as generated in the cannula.

Higher viscosities cause a further widening of the Doppler spectrum 700 because the flow flows faster in the middle and slower at the perimeter for a given volume flow of the fluid, and the areas of slow flow take up more cross-sectional area in the focus area of the ultrasonic element 210.

The Doppler frequency shifts of all velocities Vi occurring in the flow profile 225 and shown in the Doppler spectrum are:

$$Δ {f}_{i} = {f}_{0} \frac{2 {v}_{i}}{c} cos ( {a}_{i} )$$

The peak in the Doppler spectrum 700 represents the dominant velocity, or the most frequently occurring velocity analogous to a histogram. However, this value is still biased with the directional characteristic 230 of the ultrasonic element 210, which does not operate with equal sensitively in all directions.

The most frequently occurring Doppler frequency represents the most frequently occurring velocity, since the latter is to be expected due to the special mechanical design in the main direction of radiation of the ultrasonic element 210, because:

$${a}_{0 ° = 0 \to cos ( {a}_{0 °} ) = 1.}$$

For a given ultrasonic element 210 with a fixed directional characteristic 230, a width of the Doppler spectrum 700 correlates with a velocity distribution in the observation space. The detection device relies on characteristic figures of the Doppler spectrum 700 as a calculation metric-according to an exemplary embodiment based on the parameters Doppler frequency at half the maximum amplitude of the Doppler spectrum 700 and/or width of the Doppler spectrum 700, according to an exemplary embodiment at an exemplary 90% of the peak value and/or frequency of the maximum amplitude of the Doppler spectrum 700 and maximum Doppler frequency in the Doppler spectrum 700.

The calculation or the determination of the viscosity are carried out according to an exemplary embodiment by the detection device in a calculation-efficient manner using a lookup table or calibration table, abbreviated as LUT, which stores measurement data for all relevant viscosities at all relevant flow velocities. Based on the dominant Doppler frequency, a column for the dominant flow velocity is selected according to an exemplary embodiment and the viscosity is read from said column according to an exemplary embodiment based on the width of the Doppler spectrum 700. According to an exemplary embodiment, the calculation accuracy is further increased by interpolating between adjacent table entries.

A use of the flow profile 225 of the analysis device presented here for viscosity determination is demonstrated by experimentally generating different flow profiles. In an exemplary embodiment with an ultrasonic element 210, the ultrasonic element 210 is visually detectable.

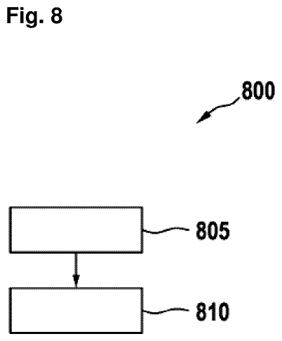

FIG. 8 shows a flow chart of a method 800 for analyzing a viscosity of a fluid according to an exemplary embodiment. This can be a method 800 that is executable by any of the analysis devices described in the figures above.

The method 800 includes detection as a step 805 and provisioning as a step 810. The detection step 805 involves determining the viscosity of the fluid using at least one Doppler parameter of a Doppler spectrum of the fluid. The provisioning step 810 involves providing or transmitting a viscosity signal that represents the viscosity determined during the detection step 805.

The method steps 805, 810 presented here can be repeated and carried out in a sequence other than that described.

1. (canceled)

2. A cardiac assist system comprising:
a flow machine;
a cannula through which a blood flow can be conveyed by the flow machine, the cannula comprising an inlet interface and configured to extend along an axis;
an ultrasonic element configured to generate a sound wave in blood, wherein the sound wave radiates through the inlet interface into the cannula along its axis and reflects off the blood; and
a detection device configured to determine a viscosity of the blood using at least one Doppler parameter of a Doppler spectrum of the sound wave reflected off the blood, wherein the detection device is configured to determine the viscosity using a functional relationship between the at least one Doppler parameter and the viscosity.

3. The cardiac assist system of claim 2, wherein the detection device is configured to determine the viscosity using a lookup table.

4. The cardiac assist system of claim 2, wherein the detection device is configured to determine the viscosity comprises using at least one cannula parameter (r) of the cannula.

5. The cardiac assist system of claim 2, wherein the detection device is configured to determine the viscosity using at least one flow parameter of a flow profile.

6. The cardiac assist system of claim 5, wherein the at least one flow parameter comprises a flow velocity (v) of a fluid through the cannula.

7. The cardiac assist system of claim 2, wherein the at least one Doppler parameter comprises a Doppler frequency or a width of the Doppler spectrum.

8. A cardiac assist system comprising:
a flow machine;
a cannula through which a blood flow can be conveyed by the flow machine, the cannula comprising an inlet interface and configured to extend along an axis;
an ultrasonic element configured to generate a sound wave in blood, wherein the sound wave radiates through the inlet interface into the cannula along the axis and reflects off the blood; and
a detection device configured to determine a viscosity of the blood using at least one Doppler parameter of a Doppler spectrum of the sound wave reflected off the blood, wherein the detection device is configured to determine the viscosity using a lookup table.

9. The cardiac assist system of claim 8, wherein the lookup table comprises values characterizing a relationship between the at least one Doppler parameter and the viscosity.

10. The cardiac assist system of claim 8, wherein the lookup table comprises an interpolation of a first viscosity and a second viscosity and wherein the detection device is configured to determine the viscosity using the interpolation of the first viscosity and the second viscosity.

11. The cardiac assist system of claim 8, wherein the detection device is configured to determine the viscosity using at least one cannula parameter (r) of the cannula.

12. The cardiac assist system of claim 8, wherein the detection device is configured to determine the viscosity using at least one flow parameter of a flow profile.

13. The cardiac assist system of claim 12, wherein the at least one flow parameter comprises a flow velocity (v) of a fluid through the cannula.

14. A cardiac assist system comprising:
a flow machine;
a cannula through which a blood flow can be conveyed by the flow machine, the cannula comprising an inlet interface and configured to extend along an axis;
an ultrasonic element configured to generate a sound wave in blood, wherein the sound wave radiates through the inlet interface into the cannula along the axis and reflects off the blood; and
a detection device configured to determine a viscosity of the blood using at least one Doppler parameter of a Doppler spectrum of the sound wave reflected off the blood, wherein the detection device is configured to determine the viscosity using the at least one Doppler parameter and wherein the at least one Doppler parameter comprises a Doppler frequency or a width of the Doppler spectrum.

15. The cardiac assist system of claim 14, wherein the detection device is configured to determine the viscosity using at least one cannula parameter (r) of the cannula.

16. The cardiac assist system of claim 14, wherein the detection device is configured to determine the viscosity using at least one flow parameter of a flow profile.

17. The cardiac assist system of claim 16, wherein the at least one flow parameter comprises a flow velocity (v) of a fluid through the cannula.

18. The cardiac assist system of claim 14, wherein the detection device is configured to determine the viscosity using a lookup table.

19. A cardiac assist system comprising:
a flow machine;
a cannula through which a blood flow can be conveyed by the flow machine, the cannula comprising an inlet interface and configured to extend along an axis;
an ultrasonic element configured to generate a sound wave in blood, wherein the sound wave radiates through the inlet interface into the cannula along the axis and reflects off the blood; and
a detection device configured to determine a viscosity of the blood using at least one Doppler parameter of a Doppler spectrum of the sound wave reflected off the blood, wherein the detection device is configured to determine the viscosity using at least one cannula parameter (r) of the cannula.

20. The cardiac assist system of claim 19, wherein the detection device is configured to determine the viscosity using at least one flow parameter of a flow profile.

21. The cardiac assist system of claim 20, wherein the at least one flow parameter comprises a flow velocity (v) of a fluid through the cannula.
